(19) United States
(12) Patent Application Publication
(10) Pub. No.: US 2024/0401083 A1
(43) Pub. Date: Dec. 5, 2024

(54) A METHOD OF ENHANCED VIRAL TRANSDUCTION USING ELECTROPORATION

(71) Applicant: MAXCYTE, INC., Rockville, MD (US)

(72) Inventors: Joan Hilly FOSTER, North Uxbridge, MA (US); James BRADY, Derwood, MD (US)

(73) Assignee: MAXCYTE, INC., Rockville, MD (US)

(21) Appl. No.: 18/694,640

(22) PCT Filed: Sep. 26, 2022
(86) PCT No.: PCT/US2022/044742

§ 371 Date: Mar. 22, 2024

Related U.S. Application Data
(60) Provisional application No. 63/261,654, filed on Sep. 24, 2021.

(51) Int. Cl. C12N 15/87; C12N 13/00; C12N 15/86

(52) U.S. Cl. CPC C12N 15/87; C12N 13/00; C12N 15/86; C12N 2750/14143

(57) ABSTRACT

Method of cell-editing comprising combining a cell or cell line with a virus, viral vector or virus like particle to form a mixture and performing simultaneous electroporation and transduction on the mixture to insert therein the virus, viral vector or virus like particle. The disclosed method simultaneously causes the virus, viral vector or virus like particle to edit, remove or modify a cell or cell line and inserting a virus, viral vector or virus like particle therein. A modified cell or cell line made by the disclosed method is also disclosed.

### PRIORITY

This application claims priority to U.S. Provisional Application No. 63/261,654 filed on Sep. 24, 2021, which is incorporated herein by reference in its entirety.

### FIELD

The present disclosure generally relates to a method of gene editing that comprises enhanced viral transduction using electroporation into a cell, specifically methods of editing genes that comprise knocking out a gene of interest and inserting a new gene and/or viral vectors via co-electroporation. The present disclosure also relates to modified cells made using this method, as well as methods of delivering a therapeutic agent to a patient comprising the modified cells.

### BACKGROUND

Electroporation is a method for loading nucleic acids into cells to achieve transfection of the loaded cells. The terminology of electroporation, electro-transfection and electroloading have been used interchangeably in the literature with emphasis on general meaning of this technology, the transgene expression and the transference of molecules into cytoplasm, respectively. Hereinafter this method of transfecting cells is referred to as electroloading that is the method using electroporation with no transfecting reagent or biologically based packaging of the nucleic acid being loaded, such as a viral vector or viral-like particle, relying only on a transient electric field being applied to the cell to facilitate loading of the cell.

Within electroporation, nucleofection is a special one involving a transfection reagent helping the transferred DNA in the cytoplasm to the nucleus. Nucleofection has been reported to transfect resting T cells and NK cells using plasmid DNA treated with a proprietary nucleofection agent (Maasho et al., 2004). It was also demonstrated that resting T cell nucleofection of chimeric receptor could lead to specific target cell killing (Finney, et al, 2004).

In addition, it is possible to load cells with mRNA that could be beneficial in respect to resting cells and cells that will be infused into a patient. First, mRNA, especially when loaded by electroloading results in minimal cell toxicity relative to loading with plasmid DNA, and this is especially true for electroloading of resting cells such as resting NK and peripheral blood mononuclear cells (PBMCs) cells. Also, since mRNA need not enter the cell nucleus to be expressed, resting cells readily express loaded mRNA. Further, since mRNA need not be transported to the nucleus, or transcribed or processed it can begin to be translated essentially immediately following entry into the cell's cytoplasm. This allows for rapid expression of the gene coded by the mRNA. Moreover, mRNA does not replicate or modify the heritable genetic material of cells and mRNA preparations typically contain a single protein coding sequence, which codes for the protein one wishes to have expressed in the loaded cell. Various studies on mRNA electroloading have been reported (Landi et al., 2007; Van De Parre et al. 2005; Rabinovich et al. 2006; Zhao et al., 2006).

A gene-editing procedure, Clustered Regularly Interspaced Short Palindromic Repeats (“CRISPR”), enables the ability to select a gene of a target location and precisely edit the gene by removing, editing, or altering a section of the DNA. CRISPR may be utilized to knock-out a gene of interest and insert a new gene and/or viral vectors. Historically, the timing of executing the insertion varies but it is always executed after the knock-out is completed. It has been shown in literature, that viral vector insertion is more efficient if executed closer in timing to the knock-out of the gene.

Electroporation disorganizes all the phospholipid membranes in the cell offering easy access into the interior of the cell including the nucleus. Technical problems scientist face is the timing of when to execute the transduction after cutting the DNA in order to maximize the efficiency and efficacy of the transduction.

Efficiency of transduction has been a critical drawback causing many programs to be delayed or discontinued due to poor therapeutic potency and lack of a streamlined manufacturing process. Electroporation is faster than standard transduction of adding the virus to a cell culture. Barlett et al. J. Virol. 2000 Mar; 74(6): 2777-2785. Current electroporation methods add a viral vector either before or after electroporation, not together. Entering the nucleus via AAV transduction is rate limiting and introducing a KO followed by a KI causes stress on the cell which decreases viability.

The present disclosure seeks to overcome one or more of the foregoing deficiencies in the prior art by a method that simultaneously co-transfects knock-out and knock-in in order to shorten the longevity of the procedure while increasing the efficiency. The claimed method provides a more streamlined manufacturing process with fewer steps and less manipulation of the cell in the process. It also increases therapeutic potency.

### SUMMARY

In one embodiment, there is disclosed a method of enhanced viral transduction using electroporation into a cell, comprising: selecting one or more cells-to-be-modified; harvesting the cells-to-be-modified; concentrating the cells-to-be-modified; combining the cells-to-be-modified with a virus, viral vector or virus like particle to form a mixture; simultaneously performing electroporation and transduction on the mixture to insert therein the virus, viral vector or virus like particle; and forming one or more co-electroporated cells.

In one embodiment, there is described a method of gene-editing, the method comprising: selecting a cell or cell line to be edited; harvesting the cell or cell line; condensing the cell or cell line by use of a centrifuge or any cell-condensing apparatus; combining the cell or cell line with a CAS9-sgRNA specimen and a viral vector coding for a desired protein or peptide to form a mixture; and performing simultaneous transfection and transduction on the mixture. The disclosed method simultaneously causes the CAS9-sgRNA specimen to edit, remove or altering a gene of interest from the cell or cell line and inserts the vector into the edited, removed or altered location of the gene.

In one embodiment, there is also disclosed a modified cell made by the disclosed method. The modified cell may be derived from blood, interstitial fluid, and tissues. No-limiting examples of the cells used in the disclosed method include cells derived from bone marrow, peripheral blood, or cord blood, or any other normal or tissues affected by a disease.

In one embodiment, the condensed cells resuspended in buffer are mixed with the virus, inserted into the processing assembly, and electroporated. In the case of a KO followed by a KI via transduction, the RNP+virus (KO) are mixed with the virus (KI) placed into the processing assembly, and electroporated.

Apart from the subject matter discussed above, the present disclosure includes a number of other exemplary features such as those explained hereinafter. It is to be understood that both the foregoing and the following descriptions are exemplary only.

### BRIEF DESCRIPTION OF THE DRAWINGS

The accompanying drawings, which comprise a part of this specification, illustrate several embodiments and, together with the description, serve to explain the principles disclosed herein. The patent or application file contains at least one drawing executed in color. Copies of this patent or patent application publication with color drawing(s) will be provided by the Office upon request and payment of the necessary fee. In the drawings:

FIG. 1 shows test results on a process according to the present disclosure that includes simultaneous electroporation and transduction, specifically a bar graph showing the percentage of cells that expressed GFP according to one embodiment of the present disclosure.

FIG. 2 shows test results on a process according to the present disclosure that includes simultaneous electroporation and transduction, specifically a bar graph showing average fluorescence per cell.

### DETAILED DESCRIPTION

As used herein, “knock-out” (abbreviated as “KO”) refers to the deletion of part of the DNA sequence or insert irrelevant DNA sequence information to disrupt the expression of a specific genetic locus.

As used herein, “knock-in” (abbreviated as “KI”) technology refers to the alteration of a DNA sequence information via a one-for-one substitution or by the addition of sequence information.

Unless specifically defined otherwise herein, all technical, scientific, and other terms used herein have the same meaning as commonly understood by one of ordinary skill in the art of ratings-based methods and web-based reputation systems and related sciences. Additional terms may be defined, as required, in the disclosure that follows.

In one embodiment, there is disclosed a method of gene-editing, that is based on executing both (KO) and (KI) at the same time. The disclosed method overcomes the deficiencies of the prior art that focused on the addition of virus before or after electroporation not together. For example, in one embodiment, the method comprises selecting a cell or cell line to be edited; harvesting the cell or cell line; condensing the cell or cell line by use of a centrifuge or any cell-condensing apparatus; combining the cell or cell line with a CAS9-sgRNA specimen and a viral vector coding for a desired protein or peptide to form a mixture; and performing simultaneous electroporation and transduction on the mixture. The disclosed method simultaneously causes the CAS9-sgRNA specimen to edit, remove or altering a gene of interest from the cell or cell line and inserts the vector into the edited, removed or altered location of the gene.

The chosen cells or cell line of interest are expanded and/or stimulated for a designated length of time pending on the cell type. On the day of electroporation, cells, suspension or adherent, are harvested and a cell sampling is taken for cell counts and viability.

In one embodiment, cells that have been cultured in the presence of serum, are washed with a buffer or basal medium to remove any residual components in the medium. The chosen cell number are condensed by centrifugation or any cell condensing apparatus pending on the scope of the experiment to the processing assembly.

In one embodiment, the correct volume of cells in buffer and/or basal medium are then mixed with the combined CAS9-sgRNA and infectivity viral units. After mixing the RNP/infectivity units with the cell pellet in buffer and/or basal medium, are inserted in the processing assembly and attached to the electroporation system.

The electroporation according to the method disclosed herein is executed. In one embodiment, the cells are then resuspended in a previously established vc/mL in complete medium which may include cytokines depending on the cell type.

Analysis is executed contingent on specific cell type program. The disclosed method is not only faster, but it is more efficient than the traditional sequential steps of transfection and transduction. For example, it has been found that greater than 20% of the co-transfected cells express the desired protein or peptide, and in some cases from 25-35% of the co-transfected cells express the desired protein or peptide. In addition, the disclosed method shows that the co-transfected cells are greater than 50% viable, even greater than 75% viable, or even greater than 90% viable.

The methods disclosed herein, may be applied to any mammalian cell line, specific blood cells, primary cells, cancer cells, diseased cells, including but not limited to any plant cell, marine cells, any eukaryotic cell types and includes other types of viral vectors.

The methods disclosed herein, may be used with a wide variety of cell populations. In some embodiments, the cells may be from blood, interstitial fluid, and any tissues, such as bone marrow, peripheral blood, or cord blood, or any other normal or tissues affected by a disease. In some embodiments, the cells may be from whole peripheral blood or whole cord blood. In some embodiments, the cells may be from whole peripheral blood mononuclear cells (PBMCs). In some embodiments, the cells may be from whole cord blood mononuclear cells (CBMCs). In some embodiments, the cells may be from a fraction of peripheral blood mononuclear cells (PBMCs). In some embodiments, the cells may be from a fraction of cord blood mononuclear cells (CBMCs). In some embodiments, the cells may be from a specific cellular component of the blood. These cells may be autologous or allogeneic to the subject receiving the cell therapy.

Non-limiting examples of PBMCs include alpha beta TCR+ T cells, gamma delta TCR+ T cells, NK cells, invariant NKT cells, B cells, dendritic cells, monocytes, macrophages, neutrophils, granulocytes, hematopoietic progenitor cells, mesenchymal progenitor cells, and stromal cells. These cells may be mature or immature cells. These cells may also be lineage committed and noncommitted cells.

In some embodiments, the isolated cells, or the cells that will be subject to modification, may be freshly isolated, previously isolated, or cryopreserved cells. In some embodiments, the modified cells may be freshly isolated, previously isolated, or cryopreserved cells. In some embodiments, the modified cells may be used immediately after modification. In some embodiments, the modified cells may be cryopreserved and used at a later time. In some embodiments, the isolated cells and/or the modified cells may be resting and unstimulated (nonactivated, nonexpanded); or activated (by antigen or stimuli); or activated, cultured, and expanded (stimulated by cytokine).

In some embodiments, the cells may be obtained from a healthy subject or diseased subject. In some embodiments, the cells may be mammalian cells. In some embodiments, the cells may be human cells, mouse cells, hamster cells. In some embodiments, the subject may be a mammal. In some embodiments, the subject may be a human, a mouse, or a hamster. In some embodiments, the mammalian cell types used are B cells (human and mouse), Vero cells, and Cardiomyocytes.

Because pathways to viral transduction are consistent between different cell types, one skilled in the art would understand that when choosing a mammalian cell type used with the disclosed method, a user would only have to make minor adjustments to the amount of virus (MOI) or the virus type, such as but not limited to AAV1 or AAV6. When choosing a virus serotype, it is beneficial to choose one that has been shown to transduce the cell of interest. Furthermore, the specific energy/voltage applied, and infective ratios will need to be optimized for each cell type.

Electroporation is a well-known method of introducing compositions into cells. Those of skill in the art are familiar with methods of electroporation. The electroporation may be, for example, flow electroporation or static electroporation. In one embodiment, the method of transfecting the cancer cells comprises use of an electroporation device as described in U.S. patent application Ser. No. 10/225,446, incorporated herein by reference. Methods and devices for electroporation are also described in, for example, published PCT Application Nos. WO 03/018751 and WO 2004/031353; U.S. patent application Ser. Nos. 10/781,440, 10/080,272, and 10/675,592; and U.S. Pat. Nos. 5,720,921, 6,074,605, 6,773,669, 6,090,617, 6,485,961, 6,617,154, 5,612,207, 7,141,425 all of which are incorporated by reference.

In some embodiments, the introducing step further comprises electroporating, wherein the spatial and temporal control of electroporation efficiency may be altered or adjusted within a population of cells. It is contemplated that various specific certain parameters can be applied to the transfecting method that would have an effect on one cell type but not on the other, such as affecting T cells rather than affecting B cells within a sample of cells from a subject.

In some embodiments, the methods and compositions disclosed herein may be effective in many immunotherapies, including, but not limited to, for the treatment of cancer and autoimmune diseases. The methods and compositions disclosed herein may also be used for treatment in several other diseases, including but not limited to, chronic diseases and infections, a viral infection, a bacterial infection, or a parasitic infection, Graft-versus-Host disease, lymphoproliferative disorders, and hyperproliferative diseases. It is contemplated that these methods and compositions may be useful for additional indications not discussed herein.

In some embodiments, the modulation is direct or indirect. In some embodiments, the alteration is direct or indirect. In some embodiments, the therapeutic effectiveness or therapeutic index may encompass an immune response, an immune activation, or an immune suppression.

In one aspect of the present disclosure, methods of generating modified cells for in vitro or ex vivo cellular vaccine therapy are provided. The methods include the steps of isolating cells, introducing a composition into the cells, and administering the cells to a subject. In some embodiments, the composition comprises at least one mRNA encoding at least one antigen, either alone or in combination thereof, wherein the modified cells may induce or are capable of inducing an immune response against the antigen. In some embodiments, the modified cells may induce or are capable of inducing an immune response against other antigens expressed by the target cell in the subject through a mechanism called epitope spreading.

In some embodiments, the gene editing agent includes CRISPR CAS-9, RNA, plasmid, mega-TALS, gene-writing, DNase I, Benzonase, Exonuclease I, Exonuclease III, Mung Bean Nuclease, Nuclease BAL 31, RNase I, 51 Nuclease, Lambda Exonuclease, RecJ, T7 exonuclease, zinc finger nuclease, meganuclease, transcription activator-like effector nuclease, or site-specific nuclease.

The term “cellular vaccines” as used herein refers to cells modified to express antigens. In particular, cellular vaccines refer to cells modified to induce immune responses against an antigen and activate immune cells against the target antigen expressing cells. The cellular vaccines if delivered to a subject and generate inflammatory milieu and elicit immune responses against malignancy, and against abnormally proliferating autoimmune cells, cells infected with viruses, bacteria, fungus, or any disease causing biological agents, thereby providing them the ability to specifically suppress and/or inactivate or kill the diseased/infected or disease causing cells. Nonlimiting examples of antigens may include proteins, polypeptides, carbohydrate antigens, lipoproteins, or peptide antigens, or peptidomimetic.

In general, molecules may include proteins, nucleotide sequences, carbohydrates, lipoproteins, or fragments thereof. Any of these molecules may be used as an antigen or used to produce an antigen, for example, in the case of the nucleotide sequence. These molecules may be natural (i.e., biological) or synthetic. In some embodiments, an antigen may be a protein, a polypeptide, a peptide multimer, a peptide avimer, a carbohydrate antigen, or a lipid protein, or a combination thereof.

The term “transduction” is used to describe a virus-mediated transfer of nucleic acids into cells. In contrast to transfection of cells with foreign DNA or RNA, no transfection reagent is needed here. The viral vector, itself, also called a virion, is able to infect cells and transport the DNA directly into the nucleus, independent of further action. After the release of DNA into the nucleus, the protein of interest is produced using the cell's machinery.

The features and advantages of the present invention are more fully shown by the following examples which are provided for purposes of illustration and are not to be construed as limiting the invention in any way.

### EXAMPLE

Human PBMCs were activated for three days with CD3/CD28 beads. Three groups of cells were transduced with AAV6-GFP according to standard protocols with 0.2, 1 and 5 multiplicities of infection (MOI). Three other sets of activated cells were suspended in MaxCyte electroporation buffer and transferred to MaxCyte 25 ul processing assemblies containing AAV at the same MOIs used for transduction. After electroporation, cells were plated at the same density as the transduced cells in media containing IL-7 and IL-15. GFP expression was assayed by flow cytometry at 24, 48 and 72 hrs.

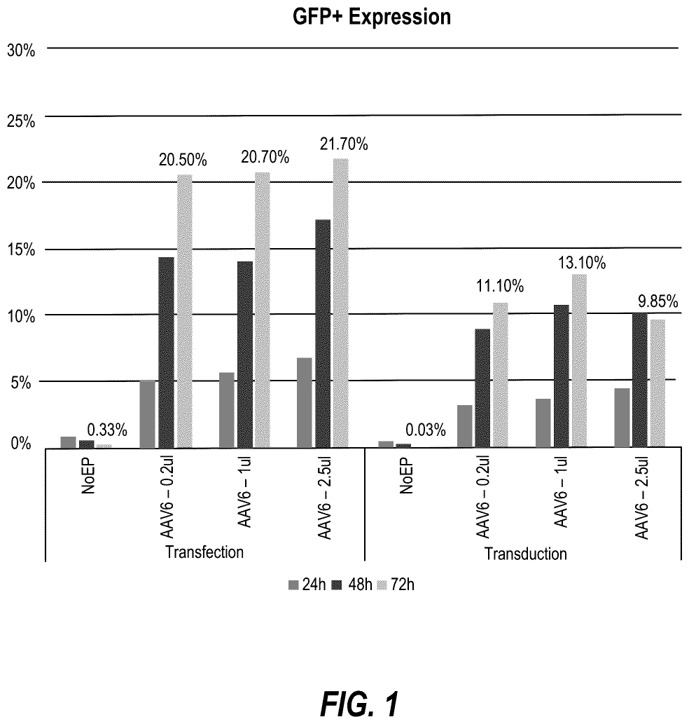

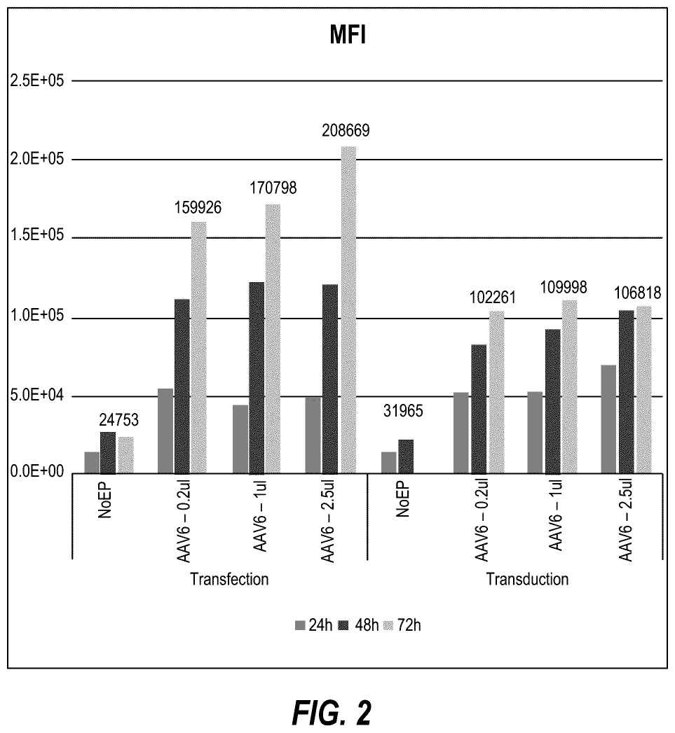

FIG. 1 shows the percentage of cells that expressed GFP. FIG. 2 shows average fluorescence per cell. Both FIGS. 1 and 2 show that simultaneous electroporation and transduction increased the number of cells taking up virus and increased the amount of virus per cell compared to standard transduction.

It is to be understood that both the descriptions disclosed herein are merely illustrative and intended to be non-limiting.

Unless otherwise expressly stated, it is in no way intended that any methods set forth herein be construed as requiring that the steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Additionally, it is contemplated that any method or composition described herein can be implemented with respect to any other method or composition described herein.

The specification and examples disclosed herein are intended to be considered as exemplary only, with a true scope and spirit of the invention being indicated in the claims. Other embodiments of the compositions, devices and methods described herein will be apparent to those skilled in the art from consideration of the disclosure and practice of the various example embodiments disclosed herein.

Other than in the examples, or where otherwise indicated, all numbers expressing quantities of ingredients, reaction conditions, analytical measurements, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

As used herein the terms “the,” “a,” or “an” mean “at least one,” and should not be limited to “only one” unless explicitly indicated to the contrary. Thus, for example, “a hybrid peptide” should be construed to mean “at least one hybrid peptide.”

All publications, patents and patent applications mentioned in this specification are herein incorporated by reference in their entirety into the specification to the same extent as if each individual publication, patent or patent application was specifically and individually indicated to be incorporated herein by reference.

What is claimed is:

1. A method of enhanced viral transduction using electroporation into a cell, comprising:
selecting one or more cells-to-be-modified;
harvesting the cells-to-be-modified;
concentrating the cells-to-be-modified;
combining the cells-to-be-modified with a virus, viral vector or virus like particle to form a mixture;
simultaneously performing electroporation and transduction on the mixture to insert therein the virus, viral vector or virus like particle; and
forming one or more co-electroporated cells.

2. The method of claim 1, wherein the virus, viral vector or virus like particle is co-electroporated with gene editing agents.

3. The method of claim 2, wherein the gene editing agents are chosen from CRISPR CAS-9, RNA, plasmid, mega-TALS, gene-writing, DNase I, Benzonase, Exonuclease I, Exonuclease III, Mung Bean Nuclease, Nuclease BAL 31, RNase I, 51 Nuclease, Lambda Exonuclease, RecJ, T7 exonuclease, zinc finger nuclease, meganuclease, transcription activator-like effector nuclease, and site-specific nuclease.

4. The method of claim 1, wherein greater than 20% of the co-electroporated cells express a desired protein or peptide.

5. The method of claim 4, wherein 25-35% of the co-electroporated cells express the desired protein or peptide.

6. The method of claim 1, wherein the co-electroporated cells have a drop in viability ranging from 25-50% compared to the cells-to-be-modified.

7. The method of claim 1, wherein the co-electroporated cells have a drop in viability no more than 20% compared to the cells-to-be-modified.

8. The method of claim 1, wherein the co-electroporated cells have a drop in viability no more than 10% compared to the cells-to-be-modified.

9. The method of claim 1, wherein the co-electroporated cells have a drop in viability no more than 5% compared to the cells-to-be-modified.

10. The method of claim 1, wherein the cells-to-be-modified is within a cell population ranging from 1×10⁵ to 1×10¹¹.

11. The method of claim 1, wherein the cells-to-be-modified is concentrated to a volume ranging from 10 μl to 1 L.

12. The method of claim 1, comprising the further step of administering the co-electroporated cells to a patient.

13. The method of claim 1, wherein the step of concentrating the cells-to-be-modified is performed with a centrifuge or any cell-condensing apparatus.

14. The method of claim 1, wherein the cells-to-be-modified are derived from blood, interstitial fluid, and tissues.

15. The method of claim 14, wherein the cells-to-be-modified are derived from bone marrow, peripheral blood, or cord blood, or any other normal or tissues affected by a disease.

16. The method of claim 14, wherein the cells-to-be-modified are derived from whole peripheral blood mononuclear cells (PBMCs) or from whole cord blood mononuclear cells (CBMCs).

17. The method of claim 16, wherein the PBMCs comprise one or more alpha beta TCR+ T cells, gamma delta TCR+ T cells, NK cells, invariant NKT cells, B cells, dendritic cells, monocytes, macrophages, neutrophils, granulocytes, hematopoietic progenitor cells, mesenchymal progenitor cells, and stromal cells.

18. The method of claim 1, wherein concentrating the cells-to-be-modified produce condensed cells that are resuspended in buffer prior to being combined with the virus, viral vector, or virus like particle.

19. A modified cell made by the method of claim 1.

20. The modified cell of claim 19, wherein the cells-to-be-modified is derived from blood, interstitial fluid, and tissues.

21. The modified cell of claim 19, wherein the cells-to-be-modified is derived from bone marrow, peripheral blood, or cord blood, or any other normal or tissues affected by a disease.

22. The modified cell of claim 21, wherein the peripheral blood and cord blood comprise peripheral blood mononuclear cells (PBMC) and whole cord blood mononuclear cells (CBMC), respectively.

23. The modified cell of claim 22, wherein the PBMC is an alpha beta TCR+ T cells, gamma delta TCR+ T cells, NK cells, invariant NKT cells, B cells, dendritic cells, monocytes, macrophages, neutrophils, granulocytes, hematopoietic progenitor cells, mesenchymal progenitor cells, stromal cells, and combinations thereof.
